(19) United States
(12) Patent Application Publication
(10) Pub. No.: US 2006/0263340 A1
(43) Pub. Date: Nov. 23, 2006

(54) Treating gastrointestinal diseases with modulators of retinoic acid

(76) Inventors: Ulrich H. von Andrian, Brookline, MA (US); Rodrigo Mora, Brookline, MA (US)

Correspondence Address: GOSZ AND PARTNERS, LLP, 450 BEDFORD STREET, LEXINGTON, MA, 02420, US

(21) Appl. No.: 11/413,874

(22) Filed: Apr. 28, 2006

Related U.S. Application Data
(60) Provisional application No. 60/676,249, filed on Apr. 29, 2005.

(51) Int. Cl. A61K 35/14; A61K 39/00; C12N 5/08; A61K 31/07

(52) U.S. Cl. 424/93.7; 435/372; 424/184.1; 514/725

(57) ABSTRACT

T cells are programmed to target the gastrointestinal tract by activation with dendritic cells capable of producing and/or transporting retinoic acid. Methods for using the programmed dendritic cells and/or T and/or B cells to treat a variety of pathogens and infectious agents residing in the intestine are also disclosed. Similarly, inhibitors of retinoic acid synthesis by dendritic cells or other cells in the gut, and inhibitors of retinoic acid receptors in T and/or B cells or other cells in the intestinal mucosa, are disclosed for treating a variety of gastrointestinal autoimmune diseases such as inflammatory bowel disease and celiac disease.

### CROSS-REFERENCE TO RELATED APPLICATIONS

This application claims the benefit of priority from U.S. Provisional Application No. 60/676,249, filed on Apr. 29, 2005, the specification of which is incorporated herein by reference.

### GOVERNMENT SPONSORED RESEARCH OR DEVELOPMENT

This work was funded in whole or in part by grants from the National Institutes of Health pursuant to Grant Nos. HL 56949; HL 54936; HL 62524; and AI 061663. The federal government may have certain rights in the invention.

### BACKGROUND OF THE INVENTION

This invention relates, in one embodiment, to a method for modifying T and/or B cells to target the cells to the gastrointestinal tract. The targeted T and/or B cells are used for the treatment of medical conditions caused by the presence of intestinal pathogens and infectious agents in the gastrointestinal tract. The pathogens and infectious agents which can be treated according to the method of the invention are, in general, those which invade, replicate, or are stored in the gastrointestinal tract and intestine mucosa, including, but not limited to, HIV pathogens. The method involves associating T and/or B cells with dendritic cells derived from gastrointestinal tissue populations, or dendritic cells that have been induced to express enzymes for converting vitamin A into retinoic acid, and/or to transport, present, or release retinoids (including retinoic acid and/or retinoic acid receptor agonists). The T and/or B cells are thereby targeted to infected areas of the gastrointestinal tract, and particularly infected areas of the small intestine. Thus, improved vaccines against pathogens and infectious agents which populate the gastrointestinal tract are prepared through the use of the targeted T and/or B cells of this invention.

This invention also relates to methods for the treatment of autoimmune diseases of the gastrointestinal tract, such as inflammatory bowel disease, through the use of inhibitors of retinoid acid-producing enzymes on intestinal epithelial cells, or for inhibiting retinoid receptors on intestinal epithelial cells, T cells, B cells, dendritic cells or dendritic cell precursors in the gastrointestinal tract. Among such autoimmune diseases are inflammatory bowel diseases. Inflammatory bowel diseases are caused when cells involved in inflammation and immune response infiltrate the lining of the gastrointestinal tract. This infiltration thickens the bowel lining and interferes with liquid absorption and motility, thereby disrupting the normal functioning of the bowel. Current therapies for treating inflammatory bowel disease focus on the use of steroids, which typically have unwanted side effects, such as inducing a predisposition to infections.

Dendritic cells are among the most powerful of the antigen-presenting cells found throughout the tissues and organs of the body. Dendritic cells are antigen presenting cells, i.e. cells that process and present antigens, and stimulate responses from naive and memory T cells. In addition to their role in antigen presentation, dendritic cells directly communicate with non-lymphoid tissue, and survey non-lymphoid tissue for injury signals (e.g., ischemia, infection, or inflammation), or for tumor growth. Once signaled, dendritic cells initiate an immune response by releasing inflammatory cytokines which trigger lymphocytes and myeloid cells. Various immunodeficiencies are believed to result from the loss of dendritic cell function.

Many infectious agents, such as HIV, are found in the gastrointestinal mucosa as a primary site for invasion or replication, or as a reservoir for such agents. Veazey et al., Science, 280, pages 427-431 (1998); Mehandru et al., Journal Exp. Med., 200, pages 761-770 (2004); Brenchley et al., Journal Exp. Med., 200, pages 749-759 (2004); Mattapallil et al.,Nature (2005). However, vaccination approaches currently being developed to treat these infectious agents and intestinal pathogens, have had only limited success. It is believed that this lack of success may be due, in part, to the lack of an effective method for efficiently targeting cellular immune responses to the intestine. See Niedergang et al., Trends Microbiology, 12, pages 79-88 (2004). This may be because percutaneous or non-oral routes of vaccine administration, such as subcutaneous or intramuscular administration, do not generate a sufficient population of T or B cells with the surface adhesion molecules necessary to promote migration of the cells into the intestine. These adhesion molecules include the integrin α4β7 molecule and the chemokine receptor CCR9. Expression of these adhesion molecules in T cells is known to be enhanced by the presence of retinoic acid. See Iwata et al., Immunity, 21, pages 527-538 (2004).

Dendritic cells are currently undergoing evaluation for therapeutic uses in clinical trials because these cells are able to generate stronger immune responses than other vaccine approaches. However, the relative effectiveness of dendritic cells as therapeutic tools has been quite limited, in part because these cells may not generate immune responses in the organs or tissues where they are most needed. Mullins et al., Journal Exp. Med., 198, pages 1023-1034 (2003); Niedergang et al., Trends Microbiology, 12, pages 79-88 (2004);

Different subsets of dendritic cells with different characteristics have been identified in intestinal lymphoid organs. All dendritic cells have the general characteristic of presenting antigens to T cells and activating the T cells. Dendritic cells derived from the intestinal mucosa, but not dendritic cells derived from peripheral tissues or the spleen, also possess the ability to induce the expression of intestine-homing molecules and gut tropism in T cells. Mora et al., Nature, 424, pages 88-93 (2003); Johansson Lindbom et al., J. Exp. Med, 198, pages 963-969 (2003); Iwata et al., Immunity, 21, pages 527-538 (2004); and Mora et al., J. Exp. Med, 201, pages 303-316 (2005).

Dendritic cells are known to have an enhancing effect on B cell proliferation, and these cells can also increase the differentiation of B cells into antibody secreting cells. Craxton et al., Blood, 101, pages 4464-4471 (2003); Jego et al., Immunity, 19, pages 225-234 (2003). Moreover, intestinal dendritic cells or retinoic acid can induce B cells to produce immunoglobulin A (the most abundant class of antibodies found in mucosal tissues). Spalding et al., J. Exp. Med, 160, pages 941-946 (1984); Tokuyama & Tokuyama, Cell. Immunol., 150, pages 353-363 (1993).

Dendritic cells from the intestinal mucosa are characterized as being capable of metabolizing food-derived vitamin A into retinoic acid. Iwata et al., Immunity, 21, pages 527-538 (2004). In addition, intestinal epithelial cells can also produce retinoic acid. Lampen et al., J. Pharmacology Exp. Ther., 295, pages 979-985 (2000).

The tissue-specific elements that stimulate dendritic cells in the intestinal mucosa to express retinoic acid-producing phenotypes are unknown. A knowledge of the factors involved in this tissue-specific stimulation may provide the opportunity to manipulate non-intestinal dendritic cells and “educate” them to impart intestinal-homing or targeting potential to the T and/or B cells upon activation. This could make the manipulated T and/or B cells an optimal vehicle for inducing intestinal-specific immune responses. Conversely, the ability to inhibit the expression of retinoic acid by intestinal dendritic cells may offer new therapeutic opportunities to treat intestinal autoimmune or hypersensitivity disorders, such as inflammatory bowel disease.

Accordingly, it is an objective of this invention to provide improved treatment methods for autoimmune diseases and hypersensitivity disorders of the gastrointestinal tract, such as inflammatory bowel disease and celiac disease, by blocking retinoic producing enzymes or retinoic acid receptors on intestinal dendritic cells.

It is also an objective of this invention to provide improved vaccines against pathogens and tumors located in the gastrointestinal tract utilizing modified T and/or B cells which target the gastrointestinal tract. The modified T and/or B cells can acquire this targeting ability by association with intestinal dendritic cells, and can thereby be used to boost vaccination protocols directed to the intestinal mucosa.

It is a further objective of this invention to confer on dendritic cells, and other antigen presenting cells, the capability of inducing intestinal targeting in T and/or B cells, and to induce intestinal-specific immune responses.

### SUMMARY OF THE INVENTION

According to one embodiment of the present invention, a method is provided for modifying T and/or B cells to target these cells to the intestinal mucosa. This method utilizes dendritic cells derived from intestinal mucosal tissues or dendritic cells otherwise induced to express enzymes for converting vitamin A into retinoic acid and/or to transport, present or release retinoids (including retinoic acid and/or retinoic acid receptor agonists). The method of this invention involves contacting the naive or effector/memory T and/or B cells with dendritic cells expressing retinoic acid-producing enzymes for a sufficient time to activate and program the T and/or B cells to target the gastrointestinal tract. When T and/or B cells are activated in the presence of these dendritic cells, the resulting effector/memory lymphocytes are programmed to migrate to the intestinal mucosa. These programmed T and/or B cells are characterized as being capable of expressing the adhesion molecules integrin α4β7 and the chemokine receptor CCR9, and by blocking the upregulation of the skin-targeting receptor on such cells, including E-selectin ligands, P-selectin ligands, and CCR4. These characteristics assist the T and/or B cells in targeting or migrating to the intestinal mucosa rather than the skin.

In another embodiment, this invention is directed to a method for modifying non-intestinal or peripheral dendritic cells, or dendritic cell precursors, to induce these cells to acquire vitamin A metabolizing ability, and/or to transport, present or release retinoids (including retinoic acid and/or retinoic acid receptor agonists) under in vitro and in vivo conditions. This method involves culturing the dendritic cells with sufficient retinoic acid for a sufficient time and under conditions sufficient to permit the dendritic cells to aquire the capacity to metabolize vitamin A and/or to transport, present or release retinoids (including retinoic acid and/or retinoic acid receptor agonists) under in vivo and in vivo conditions. Dendritic cells from non-intestinal sources, such as dendritic cells derived from the spleen or other peripheral lymphoid tissues do not express the requisite enzymes to produce retinoic acid. Such enzymes include, by way of example, retinaldehyde dehydrogenase isoforms such as RALDH-1, RALDH-2, RALDH-3, RALPH-4, and alcohol dehydrogenase isoforms, such as ADH-I, ADH-II, and ADH-III. Unmodified, non-intestinal or peripheral dendritic cells can also activate T cells, but these activated T cells are generally targeted to the skin and other peripheral tissues, but not to the intestine.

In yet another embodiment, the modified T and/or B cells of this invention can be used in vaccine formulations or protocols to target medications to the gastrointestinal tract for combating the effects of pathogens and infectious agents residing in the gastrointestinal tract. These infectious agents can be specific to the intestine, or can originate from other organs or tissues of the body, but can migrate and reside in the intestine. HIV is an example of one such infectious agent which resides in the intestinal mucosa. The vaccine formulations of this invention can include, in addition to the modified T and/or B cells, active drug ingredients directed to the specific pathogens or infectious agents of interest, and other adjuvants, excipients and additives as required or indicated in a vaccine or prophylactic formulation.

In a further embodiment, improved vaccines directed against gastrointestinal pathogens and infectious agents are described. These vaccines, which include the modified T and/or B cells of this invention, may also include active ingredients directed against specific pathogens and infectious agents, as well as adjuvants, excipients and carriers. The modified T and/or B cells are characterized by the presence of adhesion molecules which target the gastrointestinal tract. Methods of treating subjects infected with pathogens and infectious agents of the gastrointestinal tract using the vaccine compositions of this invention are also described. Typical pathogens and infectious agents that can be treated by the vaccines and methods of this invention include, but are not limited to, Human Immunodeficiency Virus (HIV), salmonella, rotavirus and poliovirus. The vaccine compositions and methods described herein are capable of efficiently and specifically boosting existing vaccine protocols by targeting the intestinal mucosa.

In a still further embodiment, the present invention is directed to a method for treating autoimmune and hypersensitivity diseases of the gastrointestinal tract by administering to a subject a pharmaceutical composition which interferes with the ability of auto-induced T and/or B cells to target the intestinal mucosa. This method involves administering to a subject a therapeutic drug that blocks retinoic acid-producing enzymes in the intestine, or that blocks retinoic acid receptors on epithelial cells, T cells, B cells, intestinal dendritic cells or dendritic cell precursors. Autoimmune diseases that can be treated using the method of this invention include, among others, celiac disease, and inflammatory bowel diseases. Prophylactic compositions for treating these diseases incorporating agents which are inhibitors of retinoic acid-producing enzymes, or which inhibit retinoic acid receptor sites on epithelial cells, T cells. B cells, dendritic cells, or dendritic cell precursors are also within the scope of this invention.

In an additional embodiment, the invention is directed to the treatment of autoimmune diseases of the skin, such as psoriasis and delayed-type hypersensitivity responses. This method involves the use of retinoids, retinoid agonists, or dendritic cells pretreated with retinoid and/or retinoid agonists, which can be injected into patients as a tolerogenic agent to block or suppress the expression of skin homing receptors. In accordance with this embodiment, a pharmaceutical composition is administered to a subject which can include, in addition to T and B cells characterized by the presence of adhesion molecules which target the skin, active ingredients directed against an autoimmune skin disease, and other additives and excipients. Diseases which may be treated using this aspect of the invention include, among others, psoriasis, atopic disorders, and delayed-type hypersensitivity responses.

The various features and advantages of the present invention will be better understood from the following specification when read in conjunction with the accompanying drawings.

### BRIEF DESCRIPTION OF THE DRAWINGS

FIG. 1 is a pictoral representation illustrating the general concept of the present invention.

FIG. 2 illustrates the experimental strategy used to treat non-intestinal dendritic cells (“DC”) with retinoic acid (“RA”), and the generation of effector T and/or B cells with RA treated or untreated DC.

FIG. 3 illustrates the effect of the retinoic acid pre-treatment of non-intestinal dendritic cells (from a D1, DC cell line derived from mouse spleen; see Winkler et al., J. Exp. Med, 185, pages 317-328 (1977)) in their capacity to induce the gut homing molecule α4β7 on T cells upon activation.

FIG. 4 illustrates the effect of retinoic acid pre-treatment of non-intestinal dendritic cells (D1-DC) in their capacity to induce the gut homing molecule CCR9 on T cells upon activation.

FIG. 5 illustrates the effect of retinoic acid pre-treatment of non-intestinal dendritic cells (D1-DC) in their capacity to induce the skin homing receptor(s) P-selectin ligands (“P-Lig”) on T cells upon activation.

FIG. 6 illustrates the effect of retinoic acid pre-treatment of non-intestinal dendritic cells (D1-DC) in their capacity to induce the skin homing receptor(s) E-selectin ligands (E-Lig) on T cells upon activation.

FIG. 7 illustrates the effect of retinoic acid pre-treatment of spleen dendritic cells in their capacity to induce gut homing molecules α4β7, CCR9 and the skin homing receptors P-selectin ligands and E-selectin ligands on T cells upon activation.

FIG. 8 illustrates the effect of retinoic acid pre-treatment of peripheral lymph node dendritic cells (PLN-DC) in their capacity to induce the gut homing molecules α4β7, CCR9 and the skin homing receptors P-selectin ligands on T cells upon activation.

FIG. 9 illustrates the biochemical steps and enzymes involved in the synthesis of retinoic acid from vitamin A (retinol).

FIG. 10 illustrates the experimental strategy used to obtain RNA from retinoic acid treated or untreated D1-DC and subsequent messenger RNA amplification by quantitative real time polymerase chain reaction (“PCR”).

FIG. 11 illustrates the effect of retinoic acid pre-treatment of non-intestinal dendritic cells (D1-DC) on mRNA expression levels of alcohol dehydrogenases (“ADHs”).

FIG. 12 illustrates the effect of retinoic acid pre-treatment of non-intestinal dendritic cells (D1-DC) on mRNA expression levels of retinaldehyde dehydrogenases (“RALDHs”).

FIG. 13 illustrates that fixed DC (D1-DC) can transport and present retinoic acid to T cells during antigen presentation.

FIG. 14 illustrates the potential for reversibility in the expression of gut homing molecules when T cells are re-activated in the presence or absence of retinoic acid.

FIG. 15 illustrates the effect of retinoic acid on T cells activated in the absence of dendritic cells by using polyclonal activation with anti-CD3 plus anti-CD28 antibodies.

FIG. 16 illustrates that effect of retinoic acid (RA) on the homing potential of B cells activated with an anti-IgM antibody.

FIG. 17 illustrates the effect of pre-treating a non-intestinal dendritic cell line (D1-DC) with intestinal (colon) and non-intestinal epithelial cell lines to induce the gut homing molecule α4β7 on T cells upon activation.

FIG. 18 illustrates that effect of vitamin A deficiency in intestinal-derived DC (from Peyer's patches) to induce the gut homing molecule α4β7 on T cells upon activation.

FIG. 19 illustrates the effect of retinoic acid pre-treatment of non-intestinal DC (D1-DC) in their capacity to induce gut homing molecules (α4β7 and CCR9) and gut migratory potential on T cells upon activation.

### DETAILED DESCRIPTION OF THE INVENTION

The dendritic cells of this invention are broadly characterized as dendritic cells which are capable of metabolizing vitamin A (retinol) into retinoid acid, and/or to transport, present or release retinoids (including retinoic acid and/or retinoic acid receptor agonists) to lymphocytes, including naïve and/or effector T and/or B lymphocytes and/or antibody secreting cells. The dendritic cells having this capability can be obtained or derived from tissues of the intestinal mucosa or from intestinal lymphoid tissue sources.

Alternatively, non-intestinal or peripheral dendritic cells, or dendritic cell precursors, can be induced to acquire the ability to metabolize vitamin A into metabolites such as retinoic acid, and/or to transport, present or release retinoids (including retinoic acid and retinoic acid receptor agonists). These non-intestinal dendritic cells do not normally metabolize vitamin A, and do not express critical vitamin A metabolizing enzymes, such as the retinaldehyde dehydrogenase isoforms. Typical retinaldehyde dehydrogenase enzymes which may be expressed by these dendritic cells include RALDH-1, RALDH-2, RALDH-3 and RALDH-4. Typical alcohol dehydrogenase isoforms which may be expressed by dendritic cells include ADH-I, ADH-II and ADH-III.

The ability to modify dendritic cells to metabolize vitamin A is also described herein. Dendritic cells are cultured in a suitable culture medium with sufficient retinoic acid for a sufficient time to induce the dendritic cells to metabolize vitamin A and/or to transport, present or release retinoids (including retinoic acid and/or retinoic acid receptor agonists). The culture medium is designed to simulate in vivo conditions in the intestinal mucosa. The dendritic cells are periodically evaluated for their capability to metabolize vitamin A and/or their capacity to induce gut homing molecules and to suppress skin homing receptors on T cells. Dendritic cells derived from peripheral lymphoid tissues or from the spleen do not normally express the requisite enzymes required to produce reinoic acid by metabolizing retinol. These non-intestinal or peripheral dendritic cells can activate T cells, but the activated T cells are not targeted to the intestine.

Activation of effector/memory or naive T and/or B cells with dendritic cells which are capable of metabolizing vitamin A into metabolites such as retinoic acid and/or to transport, present or release retinoids (including retinoic acid and/or retinoic acid receptor agonists) confers on the T cells the ability to express the surface adhesion factors integrin α4β7 and the chemokine receptor CCR9. At the same time, the upregulation of skin-homing molecules such as E-selectin ligands and P-selectin ligands on the activated T and/or B cells is effectively attenuated or blocked. Thus, the skin targeting molecules are suppressed, and the intestine targeting molecules are enhanced on T and/or B cells, imparting to the modified lymphocytes the capacity to migrate to the intestine.

These modified T and/or B cells, can be used to enhance the immune response of vaccines for intestinal ailments. A typical vaccine formulation includes the modified T cells and dendritic cells as described herein, active drug substances for combating pathogens or infectious agents residing in the gastrointestinal tract, and various additives, adjuvants, excipients and the like. These pathogens and infectious agents can be specific to the gastrointestinal tract, or they can infect other organs of the body but migrate to and reside in the intestine. Human Immunodeficiency Virus (HIV) is an example of an infectious agent residing in the intestinal mucosa and other tissues. Other examples include, but are not limited to, salmonella, rotavirus and poliovirus. A vaccine or prophylactic composition formulated to include the modified T and/or B cells and/or dendritic cells of the present invention has a boosted or enhanced immunologic response due to the presence of the modified T and/or B cells and/or dendritic cells. The preparation of suitable vaccine formulations is described herein.

Dendritic cells of the intestinal mucosa can metabolize retinol by expressing retinol-metabolizing enzymes, such as retinaldehyde dehydrogenase isoforms and alcohol dehydrogenase isoforms. As described herein, the ability to metabolize vitamin A and/or to transport, present or release retinoids (including retinoic acid and/or retinoic acid receptor agonists) can also be induced in non-intestinal dendritic cells. These dendritic cells induce T and/or B cells which express intestinal homing molecules such as α4β7 and CCR9, and such T and B cells are prevalent in the gastrointestinal tract. In autoimmune diseases, an overabundance of such T and/or B cells can cause a variety of inflammatory ailments, such as inflammatory bowel diseases and celiac disease. Inhibitors of enzymes used to metabolize vitamin A, such as the retinaldehyde dehydrogenase isoforms RALDH-1, RALDH-2, RALDH-3 and RALDH-4, and the alcohol dehydrogenase isoforms ADH-I, ADH-II and ADH-III are known to those skilled in the art. Inhibitors for these enzymes could reduce or prevent the development of these intestinal autoimmune diseases.

Consequently, this invention also embraces the treatment, prevention and amelioration of intestinal autoimmune and hypersensitivity diseases by the administration to a subject of a pharmaceutical composition containing inhibitors of enzymes capable of metabolizing vitamin A into retinal or retinoic acid, or inhibitors of retinoic acid (or retinoid) receptors, and/or agonists or enhancers of retinoid metabolism/degradation enzymes (for example, but not limited to, cytochrome P450Cyp26, P450RAI and P450RA2 enzymes). The use of such inhibitors in prophylactic compositions is expected to interfere with the ability of auto-induced T and/or B cells to target the intestinal mucosa. The pharmaceutical composition of the invention is administered to the subject in an amount sufficient to reduce or eliminate the autoimmune condition.

This invention further embraces the treatment of autoimmune skin diseases such as psoriasis and delayed hypersensitivity diseases. Retinoids, retinoid agonists, or dendritic cells pretreated with retinoids and/or retinoid agonists can be used to suppress the expression of skin traffic molecules, such as P-selectin ligands and E-selectin ligands, and skin associated chemokine receptors, while enhancing the expression of gut homing molecules α4β7 and CCR9 on lymphocytes. Such retinoids, retinoid agonists or dendritic cells can be formulated into vaccine compositions using methods known to those skilled in the art.

As used herein, the following terms and phrases shall have the following meanings unless indicated otherwise.

The term “dendritic cells” is intended to encompass both mature and immature dentritic cells, as welll as dendritic cell precursors. Dendritic cells derived from tissues of the intestinal mucosa normally possess the ability to metabolize vitamin A into retinoic acid, and/or to transport, present or release retinoids (including retinoic acid and/or retinoic acid receptor agonists), and do not require further modification for use in modifying the T and/or B cells of this invention. Dendritic cells requiring further modification in order to metabolize vitamin A and/or to transport, present or release retinoids (including retinoic acid and/or retinoic acid receptor agonists) are generally derived from non-intestinal tissue sources, such as the spleen. These latter dendritic cells are modified to express enzymes for metabolizing vitamin A into retinoic acid and/or to transport, present or release retinoids (including retinoic acid and/or retinoic caid receptor agnoists). The term “dendritic cells” may be alternatively abbreviated herein as “DC”.

A “modified”, “programmed” or “educated” T and/or B cell, as described herein, generally denotes an effector/memory or naive T and/or B cell which has been activated in the presence of a dendritic cell. Such dendritic cells may have either the ability to metabolize vitamin A into retinoic acid and/or to transport, present or release retinoids (including retinoic acid and/or retinoic acid receptor agonists), resulting in the imprinting of gut specificity, or such cells may not possess this ability (as in the case of non-intestinal dendritic cells), resulting in the induction of skin homing in T cells. The modified T and/or B cells of this invention express the surface adhesion moleucles α4β7 and CCR9, and are capable of migrating to or targeting the gastrointestinal tract when introduced into a subject.

A “subject”, as used herein, includes mammals such as human and non-human mammals. Preferred non-human mammals include primates, pigs, rodents, rabbits, canines, felines, sheep horses, and goats. Veterinary applications are within the scope of the present application.

The terms “treatment” or “treating” a medical condition includes both prophylactic and therapeutic methods of treating a subject, such as the treatment of inflammatory bowel disease, and the treatment of other autoimmune diseases of the gastrointestinal tract. “Treatment” generally denotes the administration of a therapeutic agent to a subject having a disease or disorder, a symptom of a disease or disorder, or a predisposition toward a disease or disorder, for the purpose of preventing, alleviating, relieving, reducing the symptoms of, altering, or improving the medical condition or disorder. The methods of treatment herein may be specifically modified or tailored based on a specific knowledge of the subject obtained by pharmacogenomics, and other methods for analyzing individual drug responses to therapies.

By “vaccine” is meant a prophylactic composition intended to be as used for the suppression, treatment or prevention of a disease. Vaccines can be administered orally, intravenously, intranasally, intraperitoneally or subcutaneously.

An “inhibitor” in the context of the invention generally denotes an agent that reduces or attenuates the level or the activity of enzymes capable of metabolizing vitamin A into retinal or retinoic acid, or inhibitors of retinoic acid (or retinoid) receptors, and/or agonists or enhancers of retinoid metabolism/degradation enzymes (for example, but not limited to, cytochrome P450Cyp26, P450A1 and P450A2 enzymes) in a subject. Inhibition can result from a variety of events, such as the interrupted binding of an antigen to an appropriate receptor, inactivating the enzyme, such as by cleavage or other modification, preventing or reducing the expression of a molecule on a cell, expressing an abnormal or inactive enzyme, deactivating the enzyme, preventing or reducing the proper conformational folding of the enzyme, interfering with signals that are required to activate or deactivate the enzyme, or interfering with other molecules required for the normal synthesis or functioning of the enzyme. Examples of types of inhibitors are inhibitory proteins, such as antibodies, inhibitory carbohydrates, inhibitory glycoproteins, chemical entities, and small molecules. Antibodies include humanized antibodies, chimeric antibodies, Fab₂ antibody fragments, polyclonal antibodies, and monoclonal antibodies.

A “therapeutically effective amount” of a pharmaceutical composition means that amount which is capable of treating, or at least partially preventing or reversing the symptoms of, the medical condition or disease state. A therapeutically effective amount can be determined on an individual basis and is based, at least in part, on a consideration of the species of mammal, for example, the mammal's size, the particular inhibitor used, the type of delivery system used, and the time of administration relative to the progression of the disease. A therapeutically effective amount can be determined by one of ordinary skill in the art by employing such factors and using no more than routine experimentation.

The intestinal immune system delicately maintains balances between the induction of tolerance to harmless commensal bacteria and dietary antigens, and the induction of active immunity in the face of pathogens. Because the former outnumber the latter, the immune system has a predisposition for tolerance induction. For instance, mediastinal lymph nodes and Peyer's patch cells have a cytokine profile that is dominated by IL-4 and IL-10, and have a generally immunosuppressive environment that can affect new lymphocytes. This may be because of the unusual nature of dendritic cells in the gut mucosa and associated lymphoid tissues, which illicit suppressive cytokines to stimulate, and in turn elicit, productive cytokines in non-intestinal dendritic cells.

Despite the generally suppressive environment of the intestinal immune system, the T cell response to infection is more robust and more prolonged in the intestinal mucosa than in the periphery. This suggests that another level of potent stimulation in the intestinal mucosa may override the normally tolerogenic effects of priming the gut lymphoid microenvironment. T or B cell mediated costimulation is believed to augment the mucosal response to an oral vaccine, and the lack of such costimulaton is believed to interfere with the excessive mucosal immune response for medical conditions such as inflammatory bowel disease. Thus, a potentiator of T and/or B cell activity allows the mucosa to mount an effective immune response to antigen bearing vaccines. Conversely, a suppressor or inhibitor of T and/or B cell activity ameliorates or prevents the initiation of inflammatory conditions such as inflammatory bowel disease.

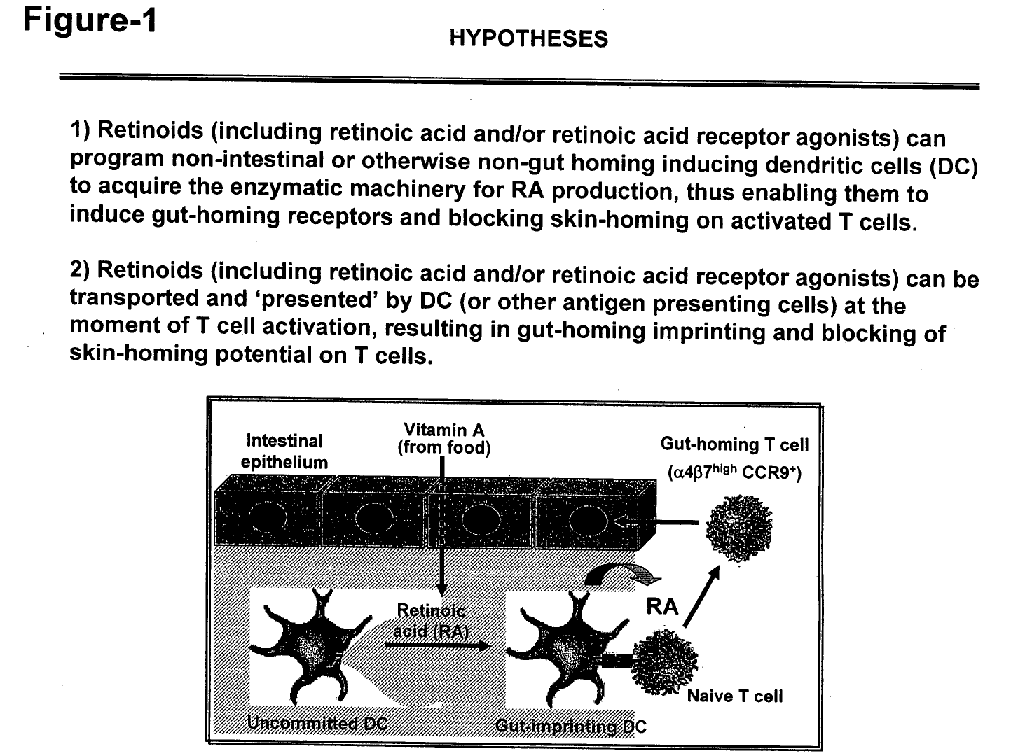

Turning now to the Figures, FIG. 1 illustrates a key hypothesis of the present invention. In brief, retinoids have the capacity to “educate” dendritic cells (DC) to synthesize retinoic acid (RA), thereby inducing gut homing and blocking skin homing capacity in T cells upon activation. On the other hand, DC can transport and “present” retinoids produced by other cells (e.g. mucosal epithelial cells) to T cells

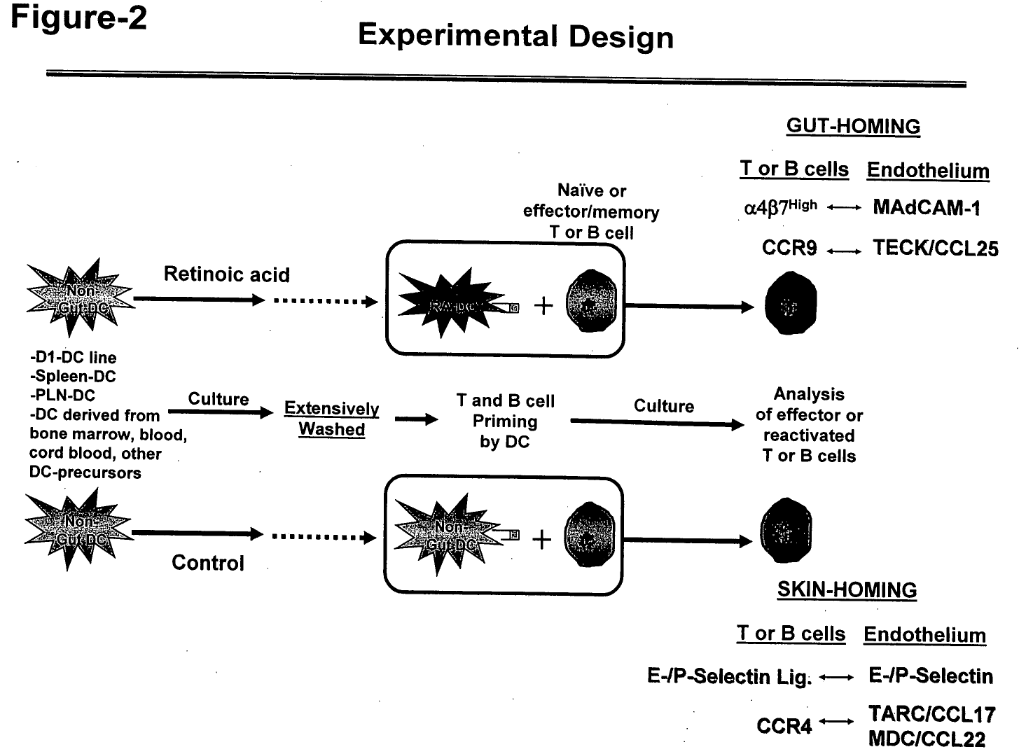

FIG. 2 illustrates the experimental strategy used to “educate” non-intestinal dendritic cells (DC) with retinoic acid (RA), and the subsequent generation of effector T and B cells using RA treated or untreated DC. Non-intestinal DC (e.g. D1-DC: a DC line derived from mouse spleen as described in Winzler et al., J. Exp. Med, 185, pages 317-328 (1997), the disclosure of which is incorporated herein in its entirety), are incubated for a sufficient time and with a sufficient concentration of RA. After that, the cells are extensively washed, loaded with antigen, and used to activate naive or effector/memory T or B cells. After 4 days, the resulting effector or reactivated T or B cells are analyzed for the expression of gut (α4β7 and CCR9) or skin (P-selectin ligands, E-selectin ligands and CCR4) homing molecules, as well as for functional properties.

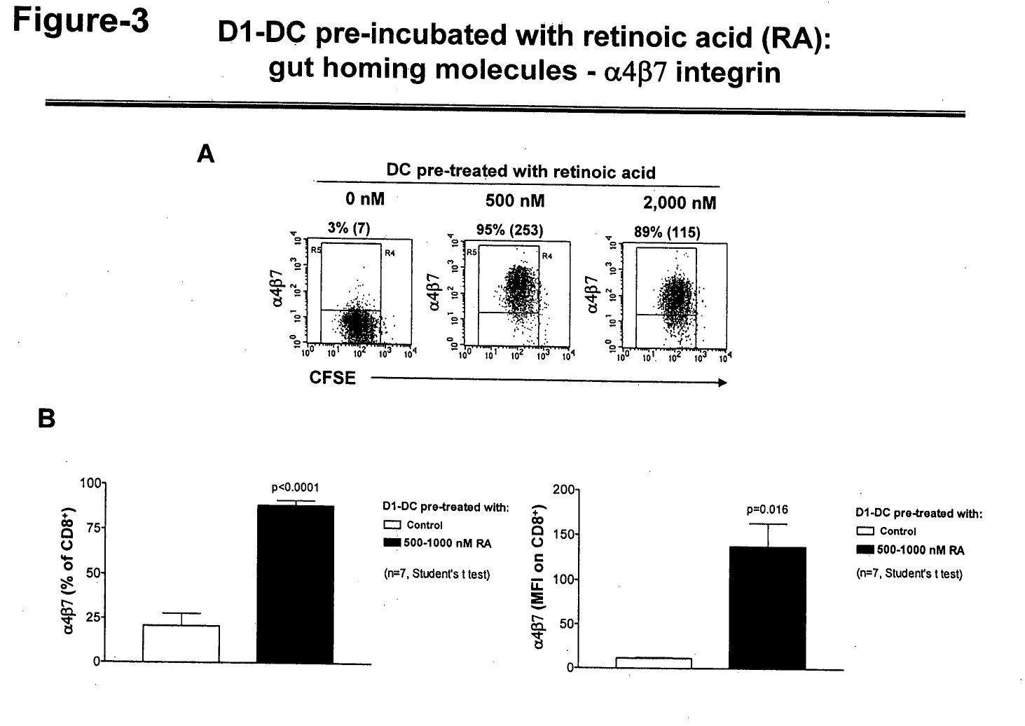

FIG. 3 illustrates the effect of retinoic acid pre-treatment of non-intestinal dendritic cells (D1-DC) in their capacity to induce the gut homing molecule α4β7 on T cells upon activation. Briefly, D1-DC are cultured with the indicated concentration of RA for 3 days. After that, DCs are extensively washed, loaded with antigen, and used to activate naïve T cells. After 4 days, the resulting effector T cells are analyzed for the expression of α4β7. (A) Flow cytometry histograms show one representative experiment (out of 7 with similar results). Numbers indicate the percentage and in parenthesis the mean intensity of cells expressing α4β7 (Y axis). Effector T cells experience an equivalent number of cell divisions as shown by the level of CFSE intensity (X axis). (B) Bar graphs show the percentage of T cells expressing α4β7 (left) and the mean fluorescence intensity (MFT) for this molecule on T cells (right). Mean±SE is from 7 independent experiments.

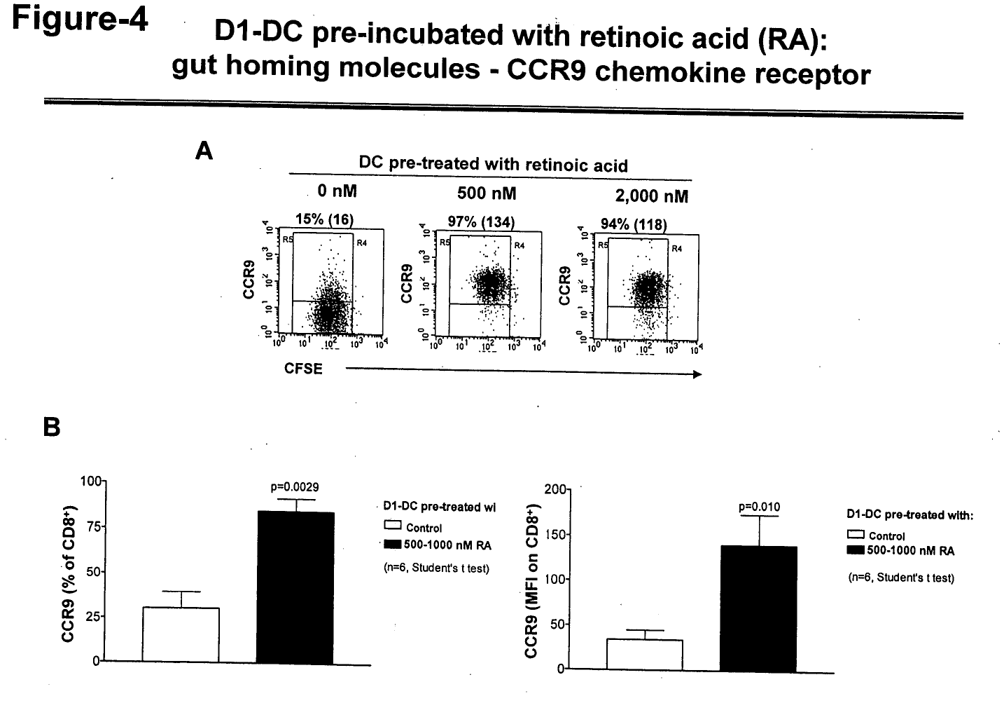

FIG. 4 illustrates the effect of retinoic acid pre-treatment of non-intestinal dendritic cells (D1-DC) in their capacity to induce the gut homing molecule CCR9 on T cells upon activation. Briefly, D1-DC are cultured with the indicated concentrations of RA for 3 days. After that, DCs are extensively washed, loaded with antigen, and used to activate naïve T cells. After 4 days, the resulting effector T cells are analyzed for their expression of CCR9. (A) Flow cytometry histograms show one representative experiment (out of 6 with similar results). The numbers indicate the percentage, and in parenthesis, the mean intensity of cells expressing CCR9 (Y axis). Effector T cells have experienced an equivalent number of cell divisions as shown by the level of CFSE intensity (X axis). (B) Bar graphs show the percentage of T cells expressing CCR9 (left) and the mean fluorescence intensity (MFI) for this molecule on T cells (right). Mean±SE from 6 independent experiments.

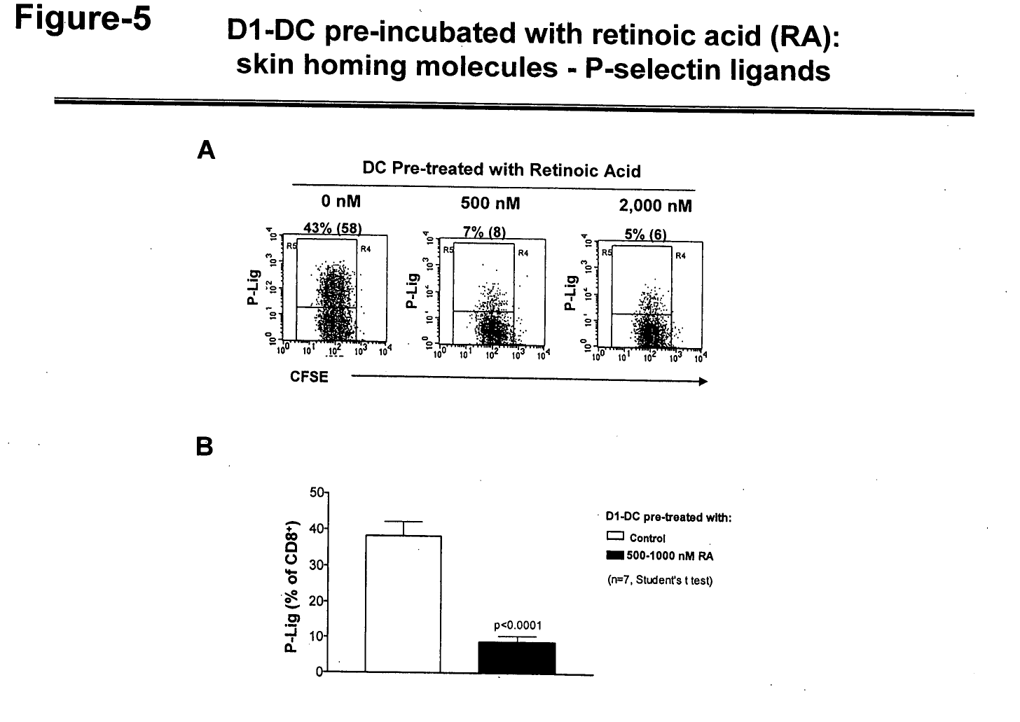

FIG. 5 illustrates the effect of retinoic acid pre-treatment of non-intestinal dendritic cells (D1-DC) in their capacity to induce P-selectin ligands (skin homing receptors) on T cells upon activation. Briefly, D1-DC are cultured with the indicated concentrations of RA for 3 days. After that, DCs are extensively washed, loaded with antigen, and used to activate naive T cells. After 4 days, the resulting effector T cells are analyzed for expression of P-selectin ligands (P Lig). (A) Flow cytometry histograms show one representative experiment (out of 7 with similar results). Numbers indicate the percentage, and in parenthesis, the mean intensity of cells expressing P Lig (Y axis). Effector T cells have experienced an equivalent number of cell divisions as shown by the level of CFSE intensity (X axis). (B) Bar graph shows the percentage of T cells expressing P Lig. Mean±SE are from 7 independent experiments.

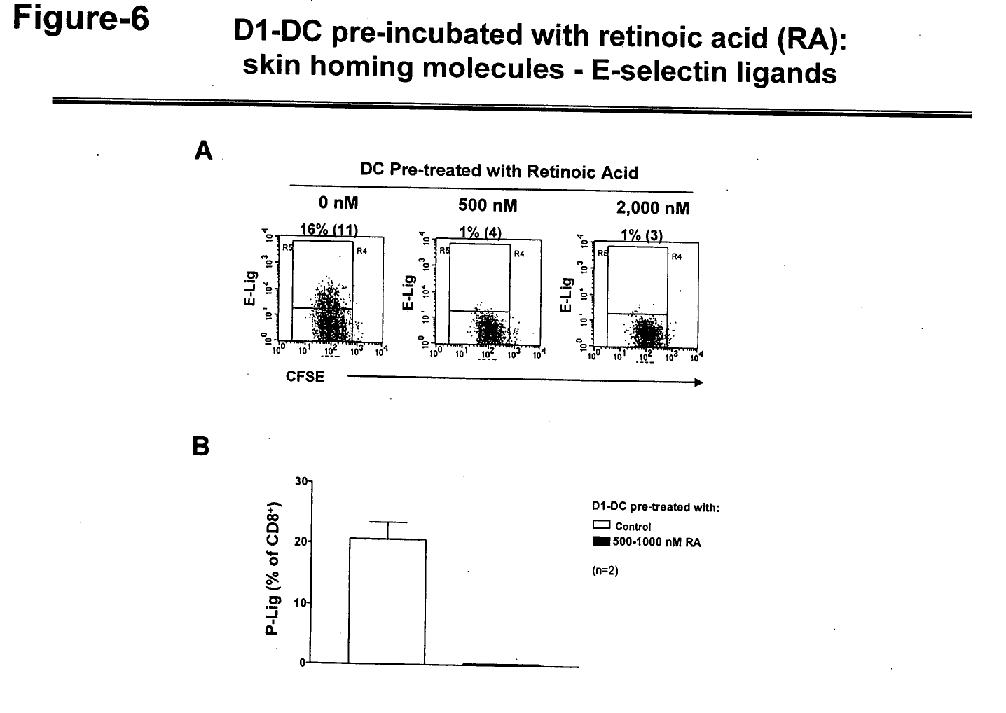

FIG. 6 illustrates the effect of retinoic acid pre-treatment of non-intestinal dendritic cells (D1-DC) in their capacty to induce E-selectin ligands (skin homing receptors) on T cells upon activation. Briefly, D1-DC are cultured with the indicated concentration of RA for 3 days. After that, DCs are extensively washed, loaded with antigen, and used to activate naive T cells. After 4 days, the resulting effector T cells are analyzed for their expression of E-selectin ligands (E Lig). (A) Flow cytometry histograms show one representative experiment (out of 2 with similar results). Numbers indicate the percentage, and in parenthesis, the mean intensity of cells expressing E Lig (Y axis). Effector T cells have experienced an equivalqnt number of cell divisions as shown by the level of CFSE intensity (X axis). (B) Bar graph shows the percentage of T cells expressing E Lig. Mean±SE are from 2 independent experiments.

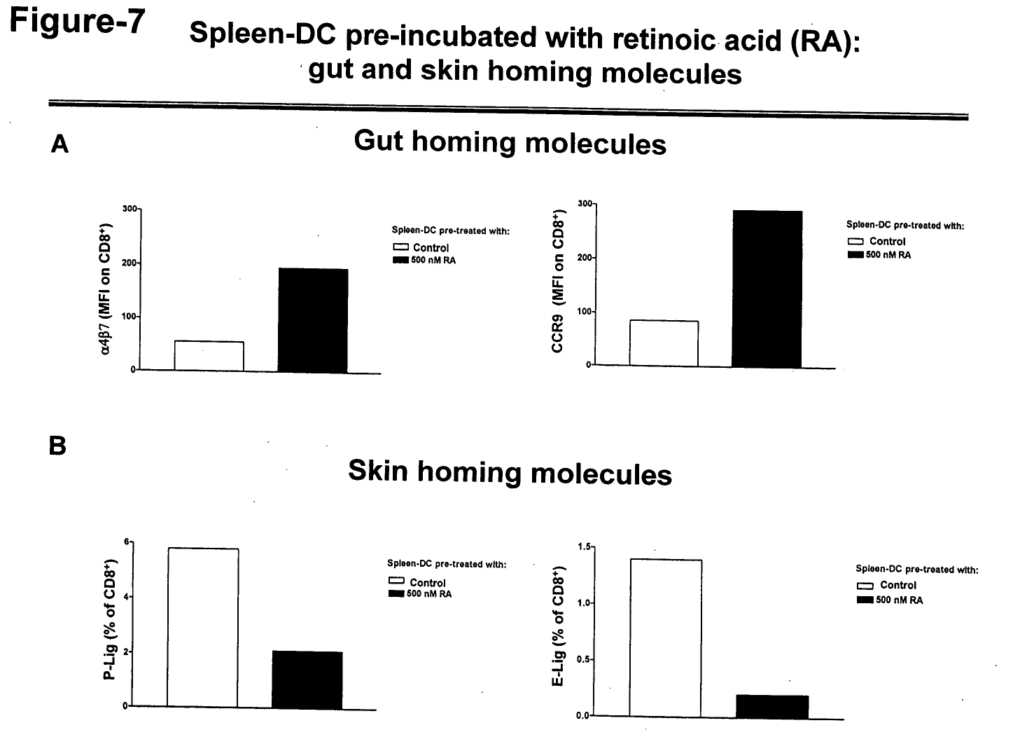

FIG. 7 illustrates the effect of retinoic acid pre-treatment of non-intestinal spleen dendritic cells in their capacity to induce (A) the gut homing molecules α4β7 and CCR9, and (B) the skin homing receptors P-selectin ligands and E-selectin ligands, on T cells upon activation. Results show one representative experiment.

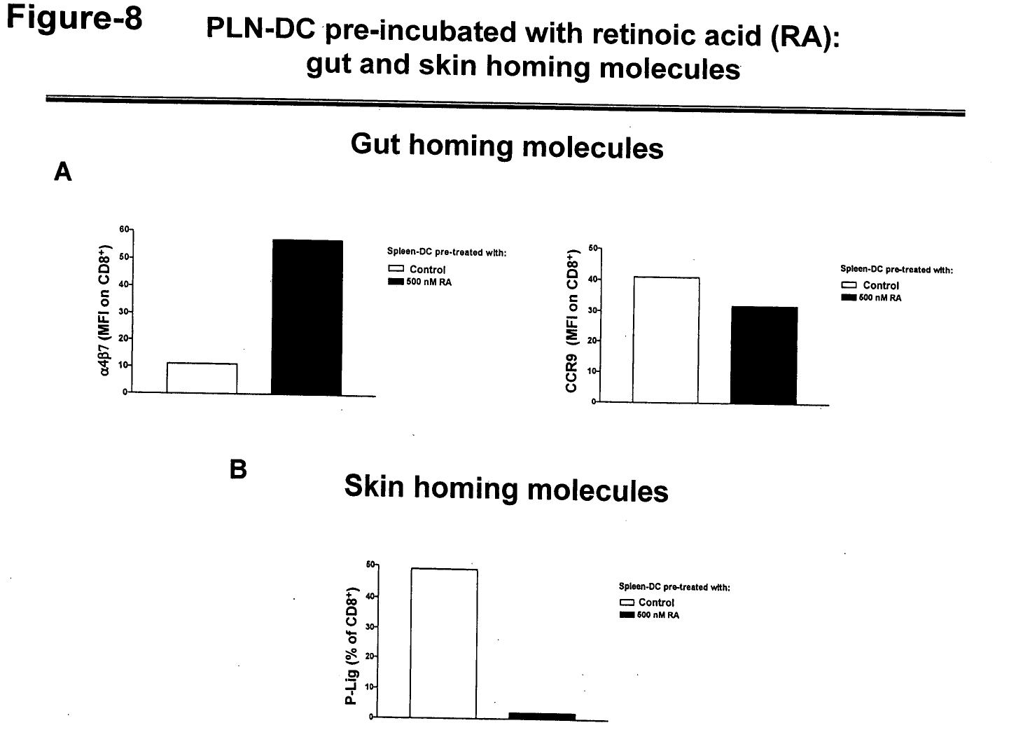

FIG. 8 illustrates the effect of retinoic acid pre-treatment of non-intestinal peripheral lymph node (PLN) dendritic cells in their capacity to induce (A) the gut homing moleucles α4β7 and CCR9, and (B) the skin homing receptors P-selectin ligands on T cells upon activation. Results show one representative experiment.

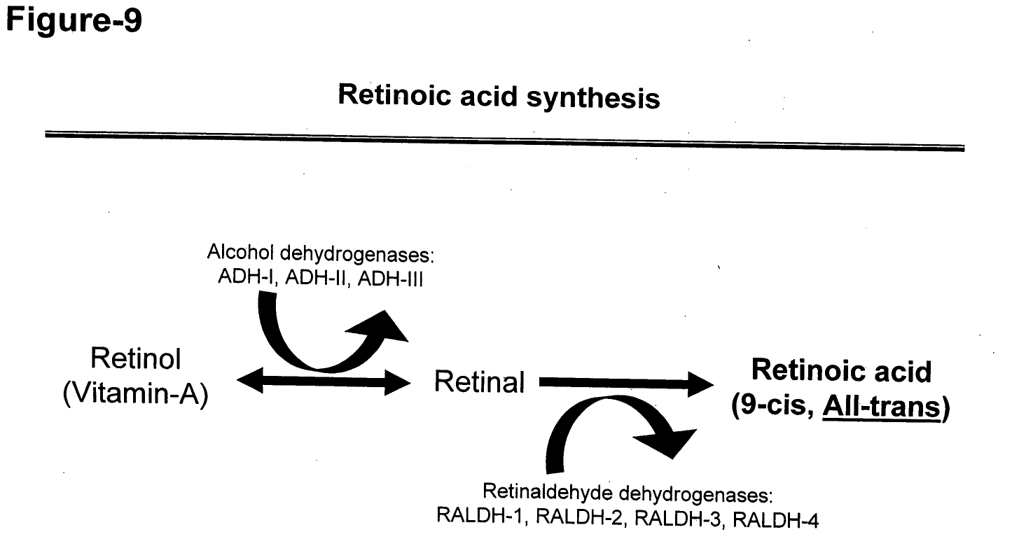

FIG. 9 illustrates the biochemical steps and enzymes involved in the synthesis of retinoic acid from vitamin A (retinol). Retinol is transformed into retinal in a reversible step catalyzed by enzymes known as alcohol dehydrogenases (ADH: at least 3 different isolforms). In an additional step, retinal is transformed into retinoic acid (9 cis or all trans isomers), and irreversible step catalyzed by enzymes known as retinaldehyde dehydrogenases (RALDH: at least 4 different isoforms).

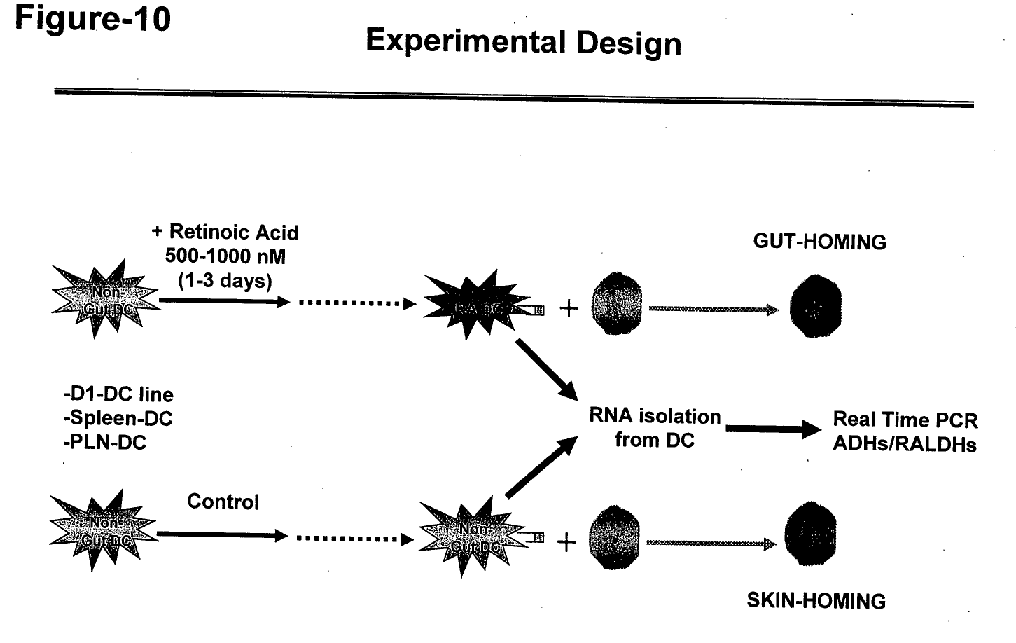

FIG. 10 illustrates the experimental strategy used to obtain RNA from retinoic acid treated or untreated D1-DC, and subsequent messenger RNA (mRNA) amplification by quantitative real time polymerase chain reaction (PCR). Briefly, D1-DC are cultured for 3-4 days with or without retinoic acid, and then total RNA is isolated from these cells. The RNA is digested with RNase free DNase, and then it is used to synthesize the complementary DNA, which is finally used to amplify the genes of interest through real time polymerase chain reaction (real time PCR).

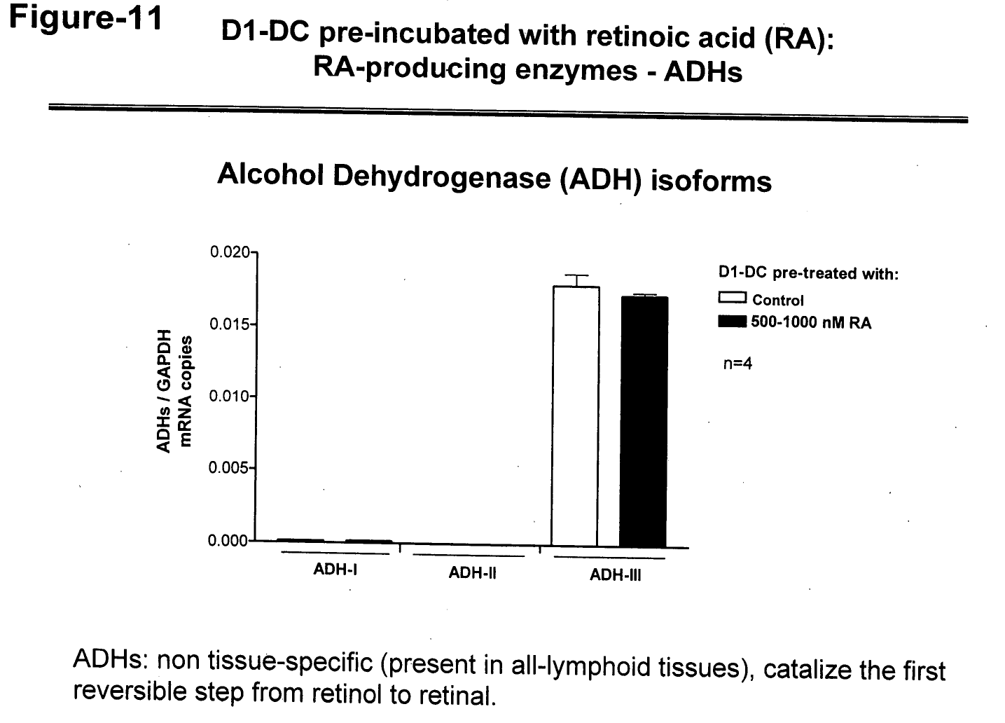

FIG. 11 illustrates the effect of retinoic acid pre-treatment of non-intestinal dendritic cells (D1-DC) in their mRNA expression levels of alcohol dehydrogenases (ADHs). RNA isolation and real time PCR amplification are performed as described in FIG. 10. For quantification purposes, mRNA for ADHs are normalized in each experiment versus the mRNA of a control gene (GAPDH: glyceraldehyde 3-phosphate dehydrogenase). Bar graph shows mean±SE from 4 independent experiments.

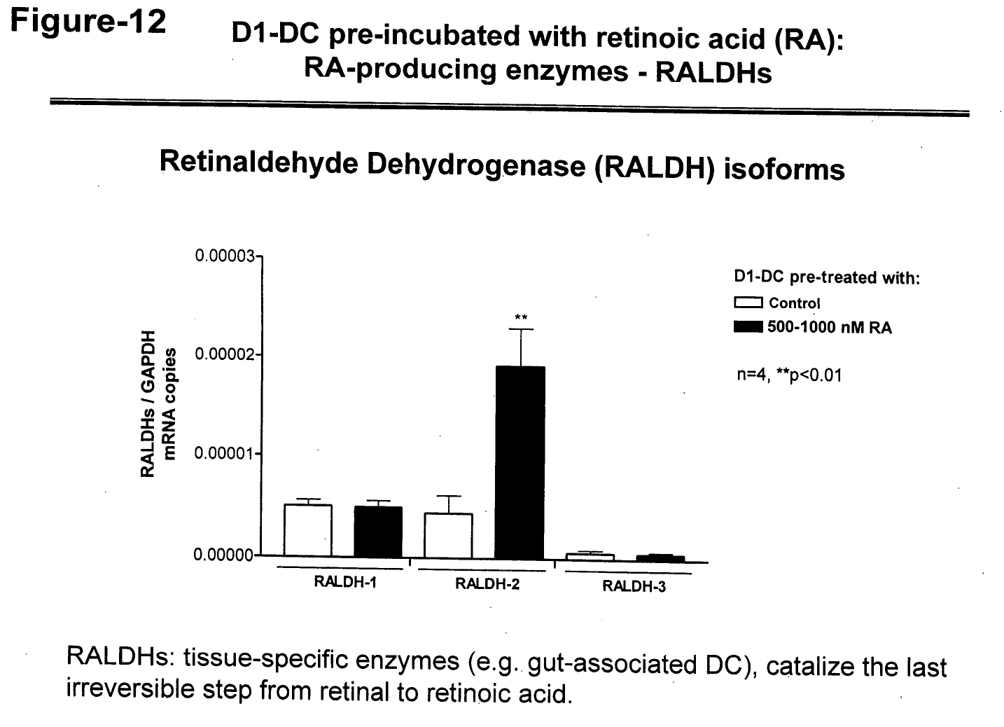

FIG. 12 illustrates the effect of retinoic acid pre-treatment of non-intestinal dendritic cells (D1-DC) in their mRNA expression levels of retinaldehyde dehydrogenases (RALDHs). RNA isolation and real time PCR amplification are performed as described in FIG. 10. For quantification purposes, mRNA for RALDHs are normalized in each experiment versus the mRNA of a control gene (GAPDH: glyceraldehyde 3-phosphate dehydrogenase). Bar graphs show mean±SE from 4 independent experiments.

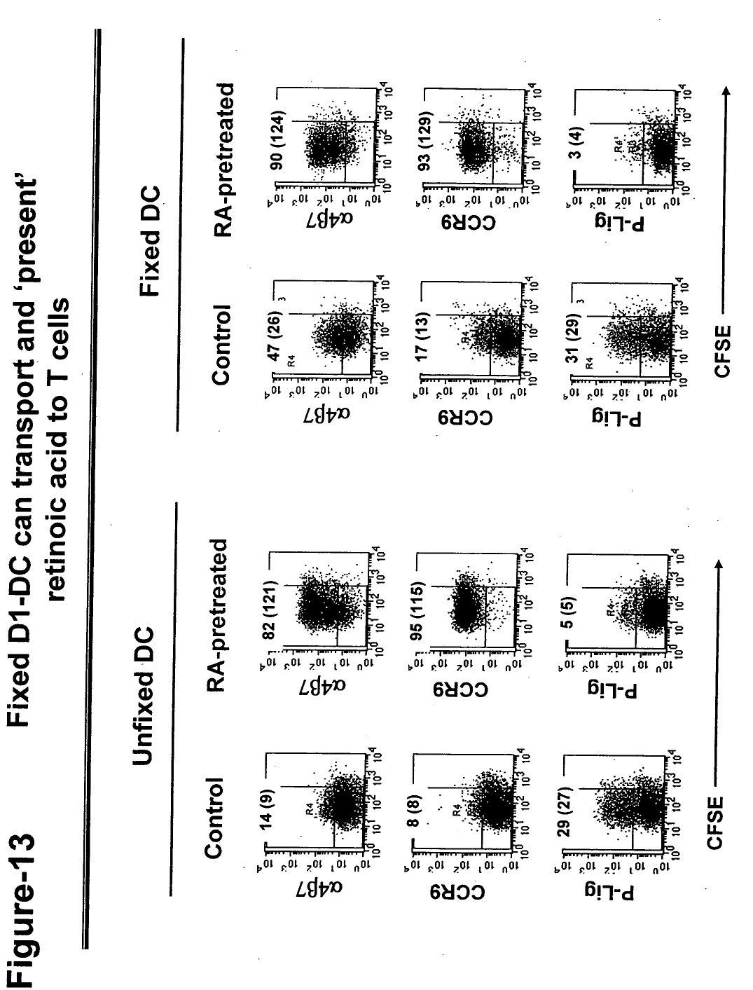

FIG. 13 illustrates that fixed DC (D1-DC) can transport and present retinoic acid to T cells during their activation. D1-DC are loaded with the specific antigenic peptide and then mildly fixed with gluteraldehyde. Fixed DC or control unfixed DC are incubated with or without retinoic acid (RA), carefully washed and then used to activate naive T cells. After 4 days, T cells are analyzed for their expression of gut homing molecules (α4β7 and CCR9) or skin homing receptors (P Lig: P-selectin ligands). Flow cytometry histograms show one representative experiment (out of 3 with similar results). Numbers indicate the percentage, and in parenthesis, the mean intensity of cells expressing the indicated homing receptor (Y axis). Effector T cells have experienced an equivalent number of cell divisions as shown by the level of CFSE intensity (X axis).

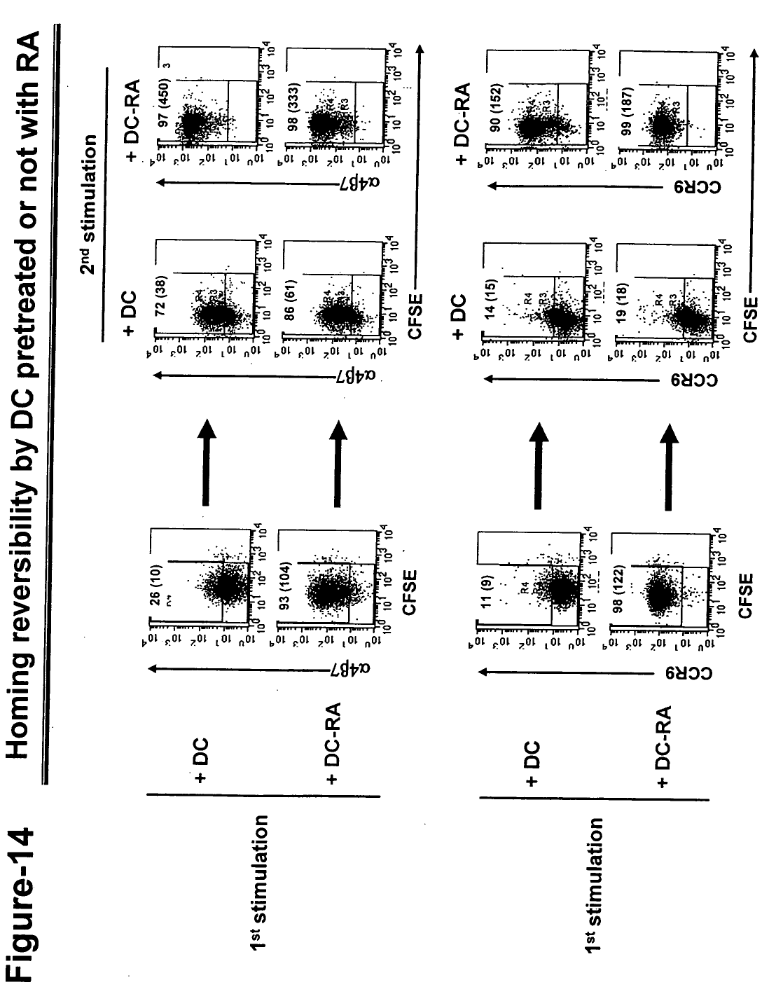

FIG. 14 illustrates the reversibility in the expression of gut homing molecules when T cells are re-activated in the presence or absence of retinoic acid (RA). Naïve T cells are activated with antigen-loaded D1-DC either incubated (DC-RA) or not (DC) with retinoic acid. After 4 days (1^{st} stimulation), effector T cells are analyzed for their expression of gut homing moleucles (α4β7 and CCR9), and then reactivated with either the same or the opposite DC. After 4 days (2^{nd} stimulation), the resulting reactivated effector T cells are analyzed for their expression of α4β7 and CCR9. Flow cytometry histograms show one representative experiment (out of 2 with similar results). The numbers indicate the percentage, and in parenthesis, the mean intensity of cells expressing the indicated homing receptor (Y axis). Effector T cells have experienced an equivalent number of cell divisions as shown by the level of CFSE intensity (X axis).

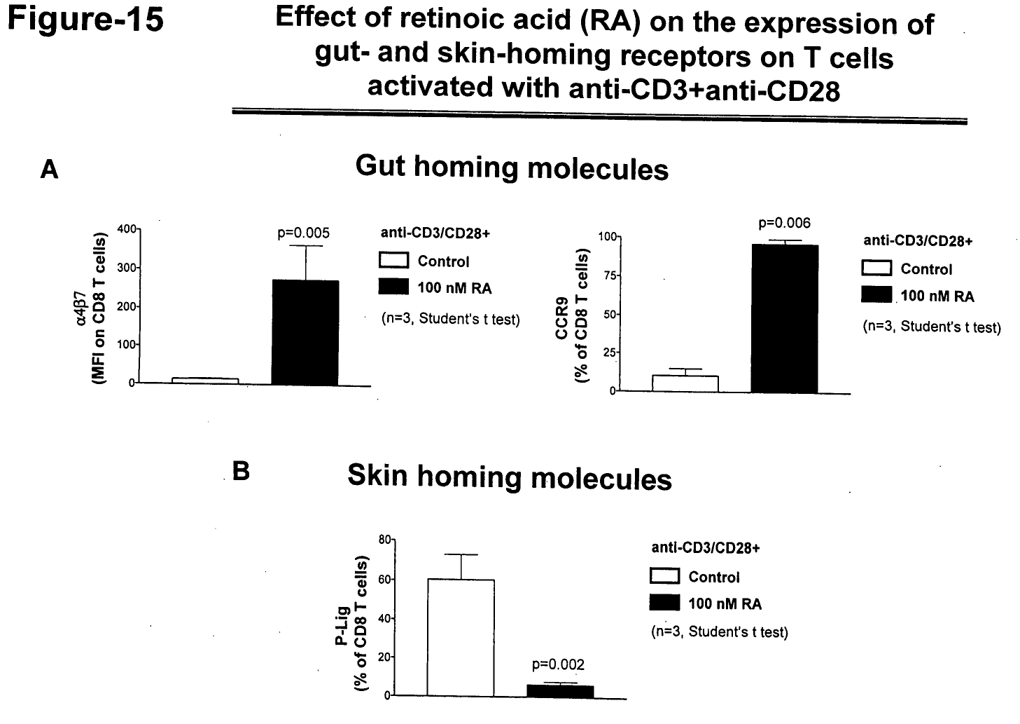

FIG. 15 illustrates the effect of retinoic acid (RA) on T cells activated with anti-CD3 plus anti-CD28 antibodies (in the absence of dendritic cells or other antigen presenting cells). Naïve T cells are activated in culture plates coated with anti-CD3 plus anti-CD28, either in the presence or the absence of RA. After 3 days, T cells are transferred into a new plate and analyzed on days 4 and 5 for the expression of (A) gut homing molecules (α4β7 and CCR9), and (B) skin homing receptors (P Lig: P selectin ligands). Bar graphs show mean±SE from 3 independent experiments.

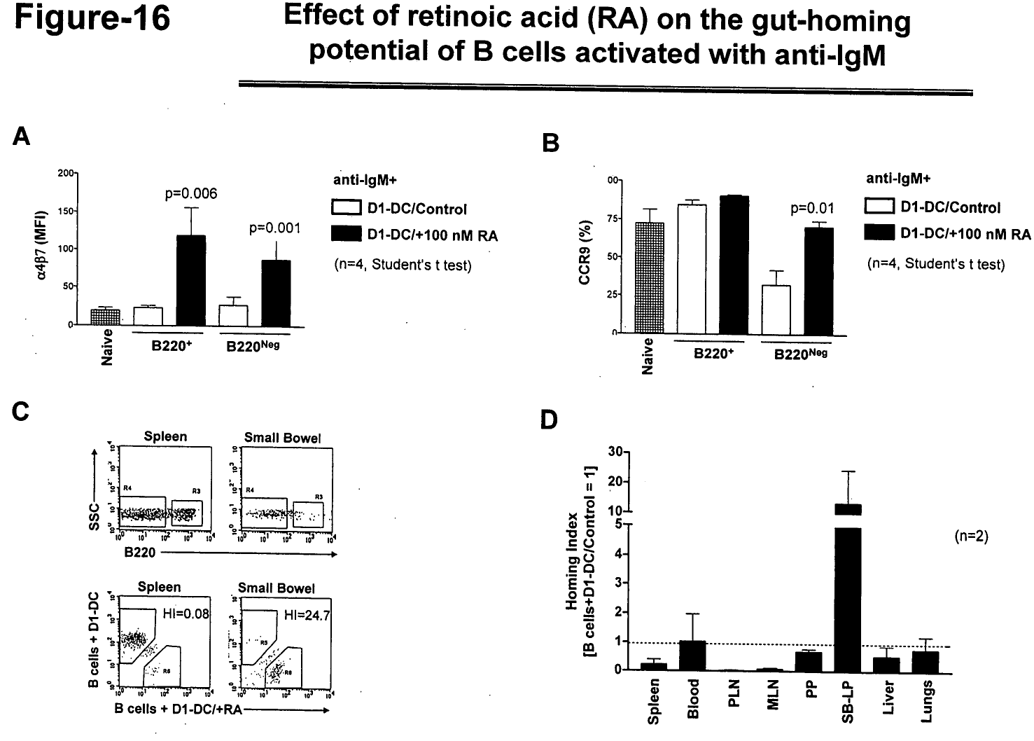

FIG. 16 illustrates the effect of retinoic acid (RA) in the homing potential of B cells activated with an anti IgM antibody. Mouse spleen B cells are activated with an anti-IgM antibody, either with or without RA, and in the presence of non-intestinal dendritic cells (D1-DC) to improve the effector B cell viability. After 4 days in culture, naïve, B220⁺ and B220^{Neg} (plasmablasts) effector B cells are assayed for (A) α4β7 (MFI: mean fluorescence intensity). (B) CCR9, and (C-D) migration of B cells activated in the presence or the absence of RA. (C) Representative homing experiments showing that B cells activated in the presence of RA localize less in the spleen and much more efficiently in the small bowel lamina propria as compared to B cells activated without RA (24 times better in this experiment). In addition, most of the cells localizing in the small bowel mucosa are B220^{Low/Neg} (plasmablasts). (D) Bar graphs showing the mean±SE of 2 independent experiments. The homing indices (HI) indicate how much more (or less) B cells activated in the presence of RA migrate with respect to those activated without RA in a given tissue (HI=1 means no difference). B cells activated in the presence of RA migrate an average more than 10 times better into the small bowel lamina propria as compared to those activated without RA. In FIG. 16: “PLN” indicates peripheral lymph nodes; “MLN” indicates mesenteric lymph nodes; “PP” is Peyer's patches; and “SB-LP” is small bowel lamina propria.

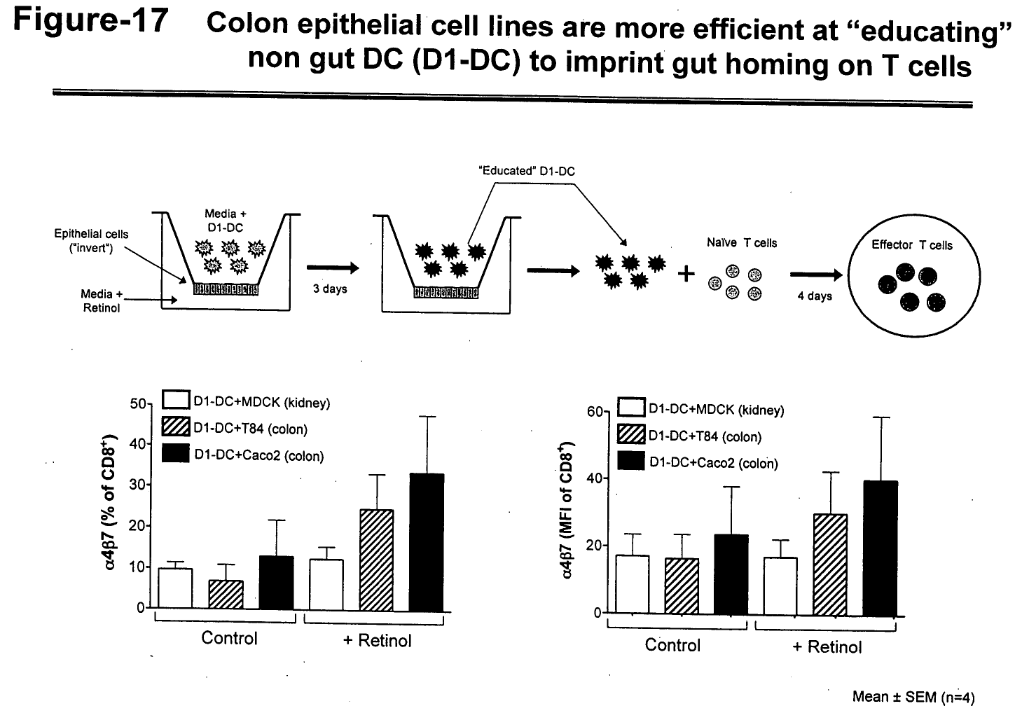

FIG. 17 illustrates the effect of pre-treating a non-intestinal dendritic cell line (D1-DC) with intestinal (colon) and non-intestinal epithelial cell lines to induce the gut homing molecule α4β7 on T cells upon activation. Intestinal epithelial cells (colon), an in vivo source of RA, or non-intestinal epithelial cells (kidney) are co-cultured with D1-DC. After 3-4 days, the “educated” D1-DC cells are collected and used to activate naïve CD8 T cells. After additional 4 days, the resulting effector T cells are analyzed for the expression of α4β7 by flow cytometry. D1-DC co-cultured with intestinal epithelial cell lines (T-84 and Caco-2) induce higher levels of α4β7 as compared to those co-cultured with a non-intestinal cell line (MDCK). This difference is apparent only when supplementing the cultures with the RA-precursor retinol (vitamin-A), suggesting that intestinal epithelial cell lines have a higher capacity to synthesize RA from retinol as compared to the non-intestinal cell line. Bar graphs show mean±SE from 4 independent experiments.

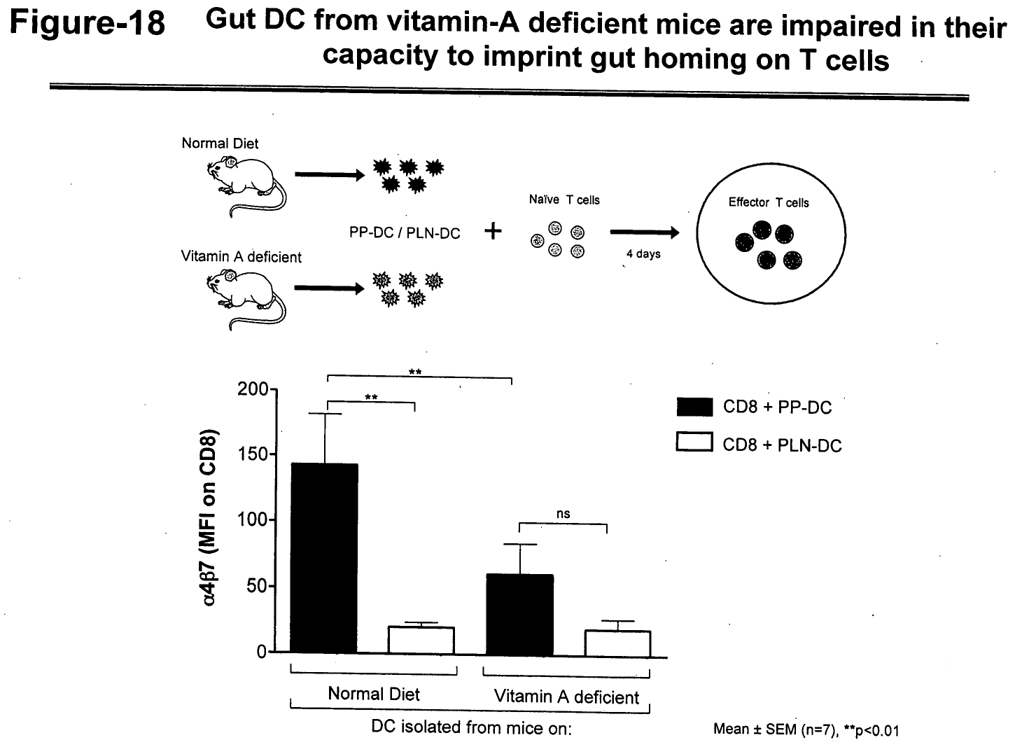

FIG. 18 illustrates the effect of vitamin A deficiency in intestinal-derived DC (from Peyer's patches) to induce the gut homing molecule α4β7 on T cells upon activation. PP-DC are isolated from mice either in normal or vitamin A deficient mice. PP-DC from vitamin A deficient mice are significantly impaired at inducing α4β7 on naïve T cells as compared to PP-DC from mice with a normal diet. Bar graphs show mean±SE from 7 independent experiments.

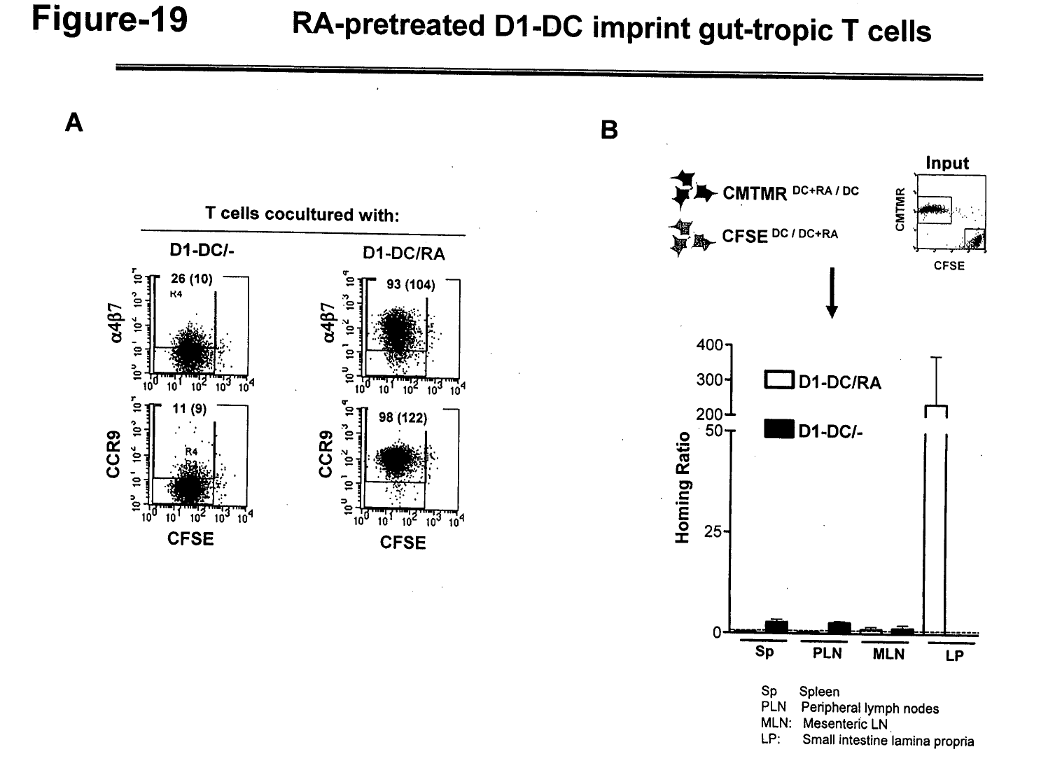

FIG. 19 illustrates the effect of retinoic acid pre-treatment of non-intestinal DC (D1-DC) in their capacity to induce (A) gut homing molecules (α4β7 and CCR9) and (B) gut migratory potential on T cells upon activation. D1-DC are cultured with RA for 3 days (as in FIG. 3). After that, DCs are extensively washed, loaded with antigen, and used to activate naive T cells. After 4 days, the resulting effector T cells are analyzed for the expression of α4β7 and CCR9. (A) Flow cytometry histograms show one representative experiment. The numbers indicate the percentage and in parenthesis the mean intensity of cells expressing α4β7 or CCR9 (Y axis). Effector T cells experience an equivalent number of cell divisions as shown by the level of CFSE intensity (X axis). (B) T cells that are activated with either D1-DC or D1-DC pretreated with RA are differentially labeled (red or green) and injected into mice to analyze their in vivo migration to different tissues. T cells activated with D1-DC pretreated with RA migrate on average 200 times better to the intestinal mucosa as compared to those activated with control D1-DC. Bar graph show the average of two independent experiments.

The modified T and/or B cells, and/or pharmacological agents that block or induce the production, function or metabolism of retinoic acid (or retinoid agonists) and its receptors, hereinafter the “active compounds(s)”, of this invention can be incorporated into pharmaceutical compositions suitable for administration to a subject. Such compositions typically comprise the active compound and a pharmaceutically acceptable carrier. As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media, and agents for pharmaceutically active substances is well known in the art. Except insofar as any conventional media or agent is incompatible with the active compound, the use thereof in the pharmaceutical compositions of the invention is contemplated. Supplementary active compounds can also be incorporated into the compositions.

The administration of the active compounds of the invention may be for either a prophylactic or therapeutic purpose. Accordingly, in one embodiment, a “therapeutically effective dose” refers to that amount of an active compound sufficient to result in a detectable change in the physiology of a recipient patient. In another embodiment, a therapeutically effective dose refers to an amount of an active compound sufficient to result in modulation of an inflammatory and/or immune response. In yet another embodiment, a therapeutically effective dose refers to an amount of an active compound sufficient to result in the amelioration of the symptoms of an inflammatory and/or immune system disorder. In a further embodiment, a therapeutically effective dose refers to an amount of an active compound sufficient to prevent an inflammatory and/or immune system response.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosages for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Generally, the therapeutically effective amount of the pharmaceutical compositions used herein will vary with the age of the subject and condition, as well as the nature and extent of the disease, all of which can be determined by one of ordinary skill in the art. The dosage may be adjusted by the physician, particularly in the event of any complication. A therapeutically effective amount will typically vary from 0.01 mg/kg to about 1000 mg/kg, preferably from about 0.1 mg/kg to about 200 mg/kg, and most preferably from about 0.2 mg/kg to about 20 mg/kg.

The present invention encompasses active agents which modulate or inhibit enzymes capable of metabolizing vitamin A into retinal or retinoic acid in vivo, retinoic acid (or retinoid) receptors, and/or retinoid metabolism/degradation (for example, but not limited to, cytochrome P450Cyp26, P450A1 and P450A2 enzymes). An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depend upon a number of factors within the knowledge of the ordinarily skilled physician, veterinarian, or researcher. The dose(s) of the small molecule will vary, for example, depending upon the identity, size, and condition of the subject or sample being treated, and also depending upon the route by which the composition is to be administered, if applicable, and the effect which the practitioner desires the small molecule to have upon the nucleic acid or polypeptide of the invention.

The modified T and/or B cells and/or dendritic cells of the invention are obtained as described herein. These T and/or B cells can be prepared from normal naive or effector/memory T and/or B cells using the dendritic cells of this invention. Alternatively, the T and/or B cells can be isolated or mobilized from tissues found in the intestinal mucosa.

Agonists and/or inhibitors of enzymes capable of metabolizing vitamin A into retinal or retinoic acid, agonists and/or inhibitors of retinoid acid (or retinoid) receptors, and agonists and/or inhibitors of retinoid metabolism/degradation enzymes (for example, but not limited to, cytochrome P450Cyp26, P450A1 and P450A2 enzymes) are known, and/or can be readily obtained by methods and techniques well known to those skilled in the art. Suitable inhibitors can also be obtained through the use of screening assays, such as high-throughput screening assays, which can be used to identify candidate inhibitors. Accordingly, a library of potentially active compounds can be prepared, and suitable inhibitory compounds within the library can be identified.

Examples of retinoid receptor agonists and of inhibitors of retinoid receptors or retinoic acid metabolism include, but are not limited to, the following:

Retinoid receptor agonists: Am80(4[(5,6,7,8 tetrahydro 5,5,8,8 tetramethyl 2-naphthalenyl)carbamoyl]benzoic acid); Am 580(4[(5,6,7,8 tetrahydro 5,5,8,8 tetramethyl 2-naphthalenyl)carboxamido]benzoic acid); LE511; PA024; HX600; HX630; HX640 and BMS189453.

Retinoid receptor antagonists: LE135; LE540(4(13H,10,11,12,13 tetrahydro 10,10,13,13,15 pentamethyldinaphtho[2,3-b][1,2-e] diazepin 7 yl) benzoic acid); LE550, 2-(arylamino)pyrimidine5-carboxylic acids (e.g. compounds PA451/6a, PA452/6b, HX531/5a).

Inhibitors of retinoic acid synthesis (e.g. RALDH inhibitors): Citral; bisdiamine [N,N octamethylenebis(dichloroacetamide)]; nitrofen; 4-biphenyl carboxylic acid; SB 210661.

See also Kagechika et al., J. Med. Chem., 31, pages 2182-2192 (1988); Tobita et al., Blood, 90, pages 967-973 (1997); Umemiya et al., J. Med. Chem., 40, pages 4222-4234 (1997); Li et al., J. Biol. Chem. 274, pages 15360-15366 (1999); White et al., Proc. Natl. Acad. Sci. USA, 97, pages 6403-6408 (2000); Vermot et la., Endocrinology, 141, pages 3638-3645 (2000); Takahashi et al., J. Med Chem., 45, pages 3327-3339 (2003); Mey et al., Am. J. Pathol., 162, pages 673-679 (2003); Iwata et al;., Immunity, 21, pages 527-538 (2004); and Keegan et al., Science, 307, pages 247-249 (2005), the disclosures of which are incorporated herein by reference in their entirety.

Exemplary doses of therapeutic compositions include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per 5 kilogram. This can be expressed as the number of cells per kilogram, if appropriate. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate the expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

In certain embodiments of the invention, a modulator or inhibitor of vitamin A metabolism and retinoic acid production, and/or retinoic acid receptor binding or signaling and/or retinoid metabolism/degradation, is administered in combination with other agents (e.g., a small molecule), or in conjunction with another, complementary treatment regime. Accordingly, the subject may be treated, for example, with an inhibitor, and further treated with an anti-inflammatory or immunosuppressive agent.

The pharmaceutical compositions can be included in a container, pack, or dispenser together with instructions for administration.

The pharmaceutical composition of the invention can include any pharmaceutically acceptable carrier known in the art. Further, the composition can include any adjuvant known in the art, e.g., Freund's complete or incomplete adjuvant. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcohol/aqueous solutions, emulsions or suspensions, including saline and buffered media. Parenteral vehicles include sodium chloride solution, Ringer's dextrose, xylitol, dextrose and sodium chloride, lactated Ringer's solution or fixed oils. Intravenous vehicles include fluid and nutrient replenishers, electrolyte replenishers (such as those based on Ringer's dextrose or xylitol), and the like. Preservatives and other additives may also be present such as, for example, antimicrobials, antioxidant, chelating agents, inert gases and the like.

The pharmaceutical compositions can be administered to the mammal by any method which allows the active compound to reach the appropriate gastrointestinal cells. These methods include, e.g., injection, infusion, deposition, implantation, oral ingestion, topical administration, or any combination thereof. Injections can be, e.g., by intravenous, intramuscular, intradermal, subcutaneous or intraperitoneal administration. Single or multiple doses can be administered over a given time period, depending upon the progression of the disease, as can be determined by one skilled in the art without undue experimentation. Administration can be alone or in combination with other therapeutic agents. The route of administration will depend on the composition of a particular therapeutic preparation of the invention, and on the intended site of action. The present compositions can be delivered directly to the site of action.

Other delivery systems can include time-release, delayed release or sustained release delivery systems. Such systems can avoid repeated administrations of the active compounds of the invention, thereby increasing the convenience to the subject and the physician. Many types of delayed release delivery systems are available and known to those of ordinary skill in the art. These include polymer-based systems such as polylactic and polyglycolic acid, polyanhydrides and polycaprolactone; nonpolymer systems include lipids such as sterols, and particularly cholesterol, cholesterol esters and fatty acids or neutral fats such as mono-, di- and triglycerides; hydrogel release systems; silastic systems; peptide based systems; wax coatings, compressed tablets using conventional binders and excipients, partially fused implants and the like. In addition, pump-based hardware delivery systems can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 clays. Long-term sustained release implants are well known to those of ordinary skill in the art and include some of the release systems described above.

With regard to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”). Pharmacogenomics thereby allows a clinician or physician to target prophylactic or therapeutic treatments to patients who will most benefit from the treatment and to avoid treatment of patients who will experience toxic drug-related side effects.

This invention is further illustrated by the following examples which should not be construed as limiting. The contents of all references, patents and published patent applications cited throughout this application, are incorporated herein by reference.

### EXAMPLE 1

### Culturing Dendritic Cells with Retinoic Acid (or Retinoid Agonists)

Dendritic cells (DC) are cultured as shown in FIG. 2. Briefly, non-intestinal DC (e.g. D1-DC: a DC cell line derived from mouse spleen (Winzler et al., J. Exp. Med., 185, pages 317-328 (1997)) is incubated in retinoic acid (RA). After that, the cells are extensively washed, loaded with specific antigen, and used to activate T cells.

In clinical settings, DC are tested for their ability to induce gut-homing molecules, or to suppress skin homing molecules, in T cellls activated using a standard mixed leukocyte reaction (MLR) assay. For this purpose, DC are generated starting from blood, bone marrow, cord blood or other source of DC precursors, and incubated with peripheral blood mononuclear cells (PBMC), or T cells derived from another donor. T cells are activated in this system by the allogeneic antigens present in the DC from the first donor. Alternatively, PBMC or T cells are stimulated using superantigens or anti-CD3/CD28 in the presence of the DC pre-incubated with RA. Importantly, PBMC or T cells are activated with DCs expanded from the same donor which have been pre-treated with RA and loaded with vaccination antigens (e.g. lysates from tumors, pathogens, etc.) or fused with tumor cells. Activated T cells are finally evaluated for their expression of gut and skin homing moleucles.

In a complementary approach, DC are assayed for their capacity to metabolize vitamin A into retinoic acid, using established procedures (see Iwata et al., Immunity, 21, pages 527-538 (2004), the disclosure of which is incorporated by reference herein in its entirety).

### EXAMPLE 2

### Imprinting T Cells (Including Regulatory T Cells) and/or B Cells (or Their Effector Progenies, Such as Memory B Cells, Plamablasts or Plasma Cells) to Target the Intestine

Dendritic cells treated with retinoic acid (RA) as described in FIGS. 3-6, or gut derived DC (e.g. mobilized from Peyer's patches or mesenteric lymph nodes), or RA itself (see FIG. 15) can be used to activate naive or effector/memory T and/or B cells. Effector/memory T and/or B cells can also be reprogrammed in their homing commitment when reactivated in a different context (see FIG. 14 and Mora et al., J. Exp. Med., 201, pages 303-316 (2005)). After 4-5 days, the resulting effector T and/or B cells are analyzed for their expression of gut or skin homing molecules and migratory behavior (see FIG. 16).

Some examples of the clinical settings where this methodology can be used include:
1. Gut homing T cells and/or B cells (and/or their corresponding effector/memory populations) can be prepared starting from peripheral blood mononulcear cells (PBMC) from a patient, and then adoptively transferred into a patient either as an independent treatment and/or to improve vaccines aimed at treating tumors having gastrointestinal locations, such as, but not limited to, enteric lymphomas, intestinal tumors and gastric tumors.
2. Gut homing T cells and/or B cells (and/or their corresponding effector/memory populations) can be prepared starting from peripheral blood mononuclear cells (PBMC) from a patient, and then adoptively transferred into a patient either as an independent treatment and/or to improve vaccines aimed at treating infections with gastrointestinal locations, such as, but not limited to, HIV, poliovirus, salmonella, and rotavirus infections.
3. Regulatory T cells (e.g. Foxp3⁺ Tregs and/or other T regulatory subsets) with gut homing capacity can be prepared starting form peripheral blood mononuclear cells (PBMC) from a patient, and then adoptively transferred into a patient either as an independent treatment and/or to improve therapies aimed at treating and/or preventing autoimmune or hypersensitivity diseases affecting the gastrointestinal mucosa, such as, but not limited to, inflammatory bowel diseases and celiac disease.

### EXAMPLE 3

### Immunization Using DC Treated with Retinoic Acid (or Retinoid Agonists)

Retinoic acid (RA) pretreated DC can be employed to immunize patients through different pathways, e.g. subcutaneously, intraperitonealy or intravenously. This can be a valuable strategy to generate or reprogram immune responses to the gut. Since retinoic acid treated DC also inhibit the expression of skin homing receptors, this approach can also be used to avoid or reprogram immune responses targeted to the skin.

Some examples of clinical settings where this methodology can be used include:
1. RA pretreated DC and/or RA or retinoid agonists can be used to formulate or to improve vaccines aimed at treating tumors with gastrointestinal locations, such as, but not limited to, enteric lymphomas, intestinal tumors and gastric tumors.
2. RA pretreated DC and/or RA or retinoid agonists can be used to formulate or to improve vaccines aimed at treating infections with gastrointestinal locations, such as, but not limted to, HIV, poliovirus, salmonella and rotavirus infections.
3. Due to their capacity to suppress skin homing receptors, RA pretreated DC and/or RA or retinoid agonists can be used to formulate or to improve treatments aimed at treating autoimmune or hypersensitivity diseases affecting the skin, such as, but not limited to, psoriasis, atopic disorders, and delayed type hypersensitivity reactions.
4. RA pretreated DC and/or RA or retinoid agonists can be used to generate gut homing regulatory T cells (e.g. Foxp3⁺ Tregs and/or other T regulatory subsets) aimed at treating and/or preventing autoimmune or hypersensitivity diseases affecting the gastrointestinal mucosa, such as, but not limited to, inflammatory bowel diseases and celiac disease.

### EXAMPLE 4

### Retinoid Antagonists Used to Block the “Education” of Dendritic Cells in the Gut and/or the Imprinting of Gut Tropism on T and/or B Cells

According to FIG. 1, retinoids can “program” and/or use DC as carriers in the intestinal mucosa and/or associated lymphoid tissues to induce gut tropism. Therefore, interfering with the generation or biological activity of retinoic acid, e.g. by interfering (ideally locally in the gut to avoid systemic effects) with the enzyme involved in the generation of retinoic acid, e.g. ADHs or RALDHs and/or with retinoic acid receptors in DC and/or lymphocytes, and/or enhancing the activity of retinoid degradation/metabolization enzymes, can be an effective strategy to block the generation of gut homing lymphocytes. This last effect would be highly desirable in the context of autoimmune or hypersensitivity disorders that effect the gut mucosa, including, but not limited to, inflammatory bowel diseases (Crohn's disease and ulcerative colitis) and graft versus host disease (GVHD).

A number of embodiments of the invention have been described herein. Nevertheless, it will be understood that various modifications may be made to the invention without departing from its spirit and scope. Accordingly, embodiments other than those specifically described herein are intended to be embraced by the following claims. Those skilled in the art will be able to ascertain, using no more than routine experimentation, many equivalents of the specific embodiments of the invention described herein. These and all other equivalents are intended to be encompassed by the following claims.

What is claimed is:

1. A method for conferring on dendritic cells which do not normally metabolize vitamin A into retinoic acid the ability to do so, said method comprising the steps of
selecting dendritic cells normally lacking the ability to metabolize vitamin A,
culturing said cells in the presence of retinoic acid (or retinoid agonists), and
evaluating said cells for (a) their ability to metabolize vitamin A and/or (b) their ability to induce gut homing and blocking skin homing receptors.

2. A method for conferring on dendritic cells which do not normally metabolize vitamin A into retinoic acid the ability to do so, said method comprising the steps of
selecting dendritic cells normally lacking the ability to metabolize vitamin A,
transfecting said cells with the genes coding for enzymes required to synthesize retinoic acid, and
evaluating said cells for (a) their ability to metabolize vitamin A and/or (b) their ability to induce gut homing and blocking skin homing receptors.

3. The method of claim 2 wherein the enzyme is selected from the group consisting of RALDH-1. RALDH-2, RALDH-3, RALDH-4, ADH-I, ADH-II and ADH-III.

4. A method for conferring on dendritic cells which do not normally transport, present or release retinoids the ability to do so, said method comprising the steps of
selecting dendritic cells normally lacking the ability to metabolize vitamin A,
culturing said cells in the presence of retinoic acid, and
evaluating said cells for their ability to induce gut homing and to blocking skin homing receptors.

5. The method of claim 4 wherein the retinoids are retinoic acid and/or retinoic acid receptor agonists.

6. The method of claim 1 wherein the dendritic cells are obtained from a source selected from the group consisting of non-lymphoid tissue, bone marrow, peripheral blood, cord blood monocytes, DC precursors, adult embryonic stem cells and the spleen.

7. A pharmaceutical composition comprising the dendritic cells obtained by the methods of claim 1 a pharmaceutically active drug substance, a pharmaceutically acceptable carrier, and an adjuvant.

8. The composition of claim 7 which is selected from the group consisting of oral formulations, subcutaneous formulations, intravenous formulations and intraperitoneal formulations.

9. A method for programming T and/or B cells to target the gastrointestinal tract comprising activating the cells in the presence of dendritic cells capable of metabolizing vitamin A and/or transporting, presenting or releasing, or contacting the T or B cells with dendritic cells which are capable of metabolizing vitamin A.

10. The method of claim 9 wherein the T and/or B cells are selected from the group consisting of regulatory T cells, naïve and/or effector/memory T and/or B cells, plasmablasts and plasma cells.

11. The method of claim 9 wherein the T and/or B cells express the molecules α4β7 and CCR9.

12. The method of claim 9 wherein the expression of skin homing receptors in said T or B cells is suppressed.

13. A pharmaceutical composition comprising the T and/or B cells of claim 9, a pharmaceutically active drug substance, a pharmaceutically acceptable carrier, and an adjuvant.

14. The composition of claim 13 which is selected from the group consisting of oral formulations, subcutaneous formulations, intravenous formulations and intraperitoneal formulations.

15. A method for treating a subject having a pathogen or infectious agent residing in the gastrointestinal tract of said subject comprising administering to the patient the pharmaceutical composition of claim 13 in an effective dosage amount.

16. A method for treating a subject having a tumor residing in the gastrointestinal tract of said subject comprising administering to the subject the pharmaceutical composition of claim 13 in an effective dosage amount.

17. A method for treating an autoimmune disease or inflammatory condition of the gastrointestinal tract comprising administering to a subject an agent capable of inhibiting an enzyme selected from the group consisting of RALDH-1, RALDH-2, RALDH-3, RALDH-4, ADH-I, ADH-II and ADH-III, and/or administering to a subject an agent capable of blocking retinoic acid receptors

18. The method of claim 17 wherein the autoimmumne disease is selected from the group consisting of inflammatory bowel diseases and celiac disease.

19. The method of claim 17 which is used as an adjunct with another therapeutic treatment.

20. A method for treating autoimmune or hypersensitivity skin diseases by immunizing a subject with dendritic cells capable of producing and/or transporting retinoic acid or retinoid agonists to block, suppress and/or reverse the acquisition of skin homing receptors on T and/or B cells.

21. The method of claim 20 wherein the disease is psoriasis or skin delayed hypersensitivity response.

22. The method of claim 9 wherein the programmed cells are regulatory T cells with intestinal-migratory potential for treating and/or preventing autoimmune or hypersensitivity diseases affecting the gastrointestinal mucosa, including inflammatory bowel diseases and celiac disease.

23. An improved vaccine formulation for treating gastrointestinal disorders comprising
a vaccine formulation directed against one or more specific pathogens or infectious agents of the gastorintestinal tract, and
dendritic cells obtained by the methods of claims 1, 2 or 4.

24. The imrpoved vaccine of claim 23 wherein the pahtogens or infectoius agents are selected form the group consisting of HIV, salmonella, rotavirus and poliovirus.

25. The vacine formulation of claim 23 which also includes adjuvants, excipients and carriers.

26. A method of boosting a vaccine formulating by targetting the vaccine to the intestinal mucos comprising combining the vaccine ex vivo with dendritic cells obtained by the methods of claim 1.
